(19) United States
(12) Patent Application Publication
(10) Pub. No.: US 2017/0281027 A1
(43) Pub. Date: Oct. 5, 2017

(54) Optical Arrangement For Power-Efficient, Low Noise Photoplethysmographic Sensor Module

(71) Applicant: Silicon Laboratories Inc., Austin, TX (US)

(72) Inventors: Moshe M. Altmejd, Austin, TX (US); Bruce P. Del Signore, Hollis, NH (US); Yahui Zhang, Austin, TX (US)

(21) Appl. No.: 15/091,205

(22) Filed: Apr. 5, 2016

(51) Int. Cl. A61B 5/024; A61B 5/00; A61B 5/0205

(52) U.S. Cl. CPC A61B 5/02427; A61B 5/02438; A61B 5/02055; A61B 5/681

(57) ABSTRACT

According to one embodiment, a power-efficient, low noise photoplethysmographic (PPG) sensor module is provided herein comprising at least one light emitting diode (LED) and at least one photodetector. The LED is generally configured for emitting light, and is positioned on the PPG sensor module for transmitting the emitted light into biological tissue. The photodetector is generally configured for detecting a portion of the light that is transmitted by the LED into the biological tissue and reflected back to the photodetector. The photodetector is an elongated photodetector having a longer dimension that is at least 1.5 times larger than a shorter dimension of the elongated photodetector. By positioning the elongated photodetector on the PPG sensor module, such that the longer dimension is facing towards the first LED, the elongated photodetector is configured to collect substantially more of the reflected light, thereby increasing the signal-to-noise ratio of the detected signal.

### BACKGROUND

### 1. Field of the Disclosure

This disclosure relates to biometric monitoring devices and, more particularly, to an optical arrangement for a power-efficient, low noise photoplethysmographic (PPG) sensor module included within a biometric monitoring device.

### 2. Description of the Relevant Art

The following descriptions and examples are provided as background only and are intended to reveal information that is believed to be of possible relevance to the present disclosure. No admission is necessarily intended, or should be construed, that any of the following information constitutes prior art impacting the patentable character of the subject matter claimed herein.

Recent advances in sensor, electronics, and power source miniaturization have allowed the size of personal health monitoring devices, also referred to herein as biometric monitoring devices, to be offered in small sizes. These biometric monitoring devices may collect and/or derive one or more types of physiological and/or environmental data from various sensors or devices embedded within the monitoring device, and may provide such data to a user interface for tracking one's health or athletic state. Example types of information that may be monitored by a biometric monitoring device include, but are not limited to, heart rate, blood pressure, oxygen saturation, respiration rate, skin and/or body temperature, calories burned, floors climbed and/or descended, light exposure, geographic location and/or heading, elevation, ambulatory speed and/or distance traveled, etc.

Heart rate monitoring, in particular, is one of the most sought after biometric sensing technologies available today for people of all fitness levels, from serious athletes looking to improve athletic performance to people simply seeking a healthier, more active lifestyle. Accurate heart rate monitoring enables precise calculation of expended calories, making it easier to maintain dieting regimens.

Heart rate measurement has traditionally been limited to the use of chest straps linked to an external device, such as a smartphone or specialized fitness watches, or to electrical touch pads integrated onto fitness equipment. These heart rate measurement solutions pose unique problems. For example, many people find chest straps inconvenient or uncomfortable to wear, while others find smartphones difficult to monitor while running or cycling and fitness watches too bulky. Since the electrical touch pads integrated onto fitness equipment are not wearable biometric monitoring devices, heart rate measurement can only be obtained from these devices while a user is in contact with the touch pads.

More recently, heart rate monitoring technology has been provided in wrist-worn biometric monitoring devices, thus providing a more convenient, comfortable way to measure heart rate as long as the user is wearing the device. However, the measurement accuracy of conventional wrist-worn monitoring devices is often adversely affected by weaker blood flow signals in the wrist (compared to chest), complex motion artifacts introduced into the measurement signal by user motion, differences in skin composition and color, and/or poorly designed sensor modules. Conventional wrist-worn monitoring devices also tend to be costly and power hungry, which limits the battery life of such devices. Some wrist-worn biometric monitoring devices are also bulky and cumbersome to wear.

Therefore, a need exists for an improved biometric monitoring device that provides accurate heart rate monitoring in a space-constrained wearable device, while reducing power consumption and extending battery life. Although described herein as a wearable biometric monitoring device, and more specifically, a wrist-worn biometric monitoring device, the sensor modules described herein could be used in a variety of wearables, including activity-tracking fitness bands and straps, pedometers and smart watches, as well as fitness equipment, bathroom scales and patient monitoring devices.

### SUMMARY

The following description of various embodiments of a photoplethysmographic (PPG) sensor module and a biometric monitoring device comprising the same is not to be construed in any way as limiting the subject matter of the appended claims.

Generally speaking, the present disclosure is believed to provide an improved biometric monitoring device that provides accurate heart rate monitoring in a space-constrained wearable device, while reducing power consumption and extending battery life. The present disclosure achieves these objectives by providing the biometric monitoring device with an improved PPG sensor module, which optimizes the optical arrangement of one or more light emitting diode(s) and one or more photodetector(s) included within the PPG sensor module, so as to maximize the amount of reflected light that can be received from a biological tissue and detected by the photodetector(s). The optimized arrangements disclosed herein improve the signal-to-noise ratio (SNR) of the detected signal and increase the accuracy of the heart rate measurement signal, enabling heart rate to be accurately monitored, even in peripheral locations with weaker blood flow. Since SNR is improved, power consumption may be reduced in the PPG sensor modules described herein without adversely affecting the measurement accuracy. In some embodiments, accuracy may be further improved by providing a plurality of LED/photodetector pairs within the PPG sensor module, each pair potentially providing a different signal path for receiving the reflected light from a biological tissue. This provides signal diversity and may significantly improve measurement accuracy for a variety of different skin types and measurement locations (e.g., wrist, upper arm, ankle, etc.) by enabling the reflected signal with the best SNR to be used for monitoring heart rate. Although described herein for monitoring heart rate, the PPG sensor modules described herein can also be used to monitor respiration and other circulatory conditions, such as blood pressure, blood volume, arterial stiffness, oxygen saturation, blood glucose levels, cardiac rhythm, hypovolemia and hypervolemia, among others.

According to one embodiment, an improved PPG sensor module may generally comprise a first light emitting diode (LED) and a photodetector. The first LED may be configured for emitting light, and may be positioned on the PPG sensor module for transmitting the emitted light into biological tissue. The photodetector may generally be configured for detecting a portion of the light that is transmitted by the first LED into the biological tissue and reflected back to the photodetector.

In general, the first LED may be configured for emitting light within substantially any visible or infrared wavelength range. When monitoring heart rate, however, the first LED may be configured for emitting green light within a wavelength range extending between about 495 nm and about 570 nm. In some embodiments, the improved PPG sensor module may include one or more additional LEDs, which are configured for emitting green light, blue light (e.g., light within a wavelength range extending between about 380 nm and 450 nm), red light (e.g., light within a wavelength range extending between about 620 nm and 750 nm), and/or infrared light (e.g., light in a wavelength range extending between about 700 nm and 1 mm). When light emitted by the one or more additional LEDs is transmitted into the biological tissue, reflected back and received by the photodetector, the reflected/received light may be used to provide signal diversity for the heart rate measurement signal, remove noise components in the green light signal transmitted by the first LED and detected by the photodetector, or collect and/or derive data pertaining to other physiological conditions.

The photodetector is preferably an elongated photodetector having a longer dimension that is at least 1.5 times larger than a shorter dimension of the elongated photodetector. In some embodiments, the longer dimension of the elongated photodetector may be approximately 2 to approximately 8 times larger than the shorter dimension of the elongated photodetector. The elongated photodetector is positioned on the PPG sensor module, such that the longer dimension is facing the first LED. In some embodiments, the elongated photodetector may be positioned on the PPG sensor module, such that a line passing through a center point of the first LED in a plane of the PPG sensor module substantially bisects the longer dimension of the elongated photodetector. Arranging an elongated photodetector in such a manner may maximize the collection angle of reflected light that can be detected by the elongated photodetector, so that more of the reflected light can be collected along its longer edge.

In some embodiments, the PPG sensor module may further comprise an additional elongated photodetector, which is configured for detecting a portion of the light that is transmitted by the first LED into the biological tissue and reflected back to the additional elongated photodetector. Like the first elongated photodetector, a longer dimension of the additional elongated photodetector is preferably at least 1.5 times larger (and more preferably, about 2 to about 8 times larger) than a shorter dimension of the additional elongated photodetector. The first LED is arranged between the elongated photodetector and the additional elongated photodetector. In some embodiments, the additional elongated photodetector may be positioned on the PPG sensor module, such that a line passing through a center point of first LED in the plane of the PPG sensor module substantially bisects the longer dimension of the elongated photodetector and the longer dimension of the additional elongated photodetector. In some embodiments, signal diversity may be provided by separating each LED/photodetector pair by a different distance. For example, a first distance separating the elongated photodetector and the first LED may be substantially greater than or substantially less than a second distance separating the first LED and the additional elongated photodetector. In order to be “substantially greater than” or “substantially less than,” the first distance may be approximately 2 to approximately 16 times larger or smaller than the second distance.

In some embodiments, the PPG sensor module may further comprise a second LED, which is configured for emitting light and positioned on the PPG sensor module for transmitting the emitted light into the biological tissue. In some embodiments, the elongated photodetector may be positioned between the first and second LEDs, such that a line passing through a center point of the first LED and a center point of the second LED in a plane of the PPG sensor module substantially bisects the longer dimension of the elongated photodetector. In other words, the elongated photodetector may be positioned between the first and second LEDs, such that a line passing through a respective center point of the first and second LEDs also passes through the center point of the elongated photodetector.

In some embodiments, the PPG sensor module may further comprise a third LED and a fourth LED, each of which may be configured for emitting light and may be positioned on the PPG sensor module for transmitting the emitted light into the biological tissue. In some embodiments, the elongated photodetector may be positioned between the first LED and the second LED, such that a line passing through a respective center point of the first and second LEDs in the plane of the PPG sensor module passes through a top half portion of the longer dimension of the elongated photodetector. The elongated photodetector may also be positioned between the third LED and the fourth LED, such that a line passing through a respective center point of the third and fourth LEDs in the plane of the PPG sensor module passes through a bottom half portion of the longer dimension of the elongated photodetector.

According to another embodiment, an improved PPG sensor module may generally comprise a first LED and a second LED, each configured for emitting light and positioned on the PPG sensor module for transmitting the emitted light into biological tissue, and a photodetector which is configured for detecting a portion of the light, which is transmitted by at least one of the first and second LEDs into the biological tissue and reflected back to the photodetector. As in previous embodiments, the photodetector is preferably an elongated photodetector having a longer dimension, which is at least 1.5 times larger (and more preferably about 2 to about 8 times larger) than a shorter dimension of the elongated photodetector.

In this embodiment, the photodetector is preferably arranged on the PPG sensor module between the first and second LEDs, such that a first distance separating the photodetector and the first LED is substantially greater than or substantially less than a second distance separating the photodetector and the second LED. For example, the first distance may be approximately 2 to approximately 16 times greater than or less than the second distance. By separating the first LED/photodetector pair and the second LED/photodetector pair by a substantially different distance, the improved PPG sensor module may provide signal diversity by providing different signal paths for the reflected light to return to the photodetector.

In some embodiments, the PPG sensor module may further comprise a third LED and a fourth LED, each of which may be configured for emitting light and may be positioned on the PPG sensor module for transmitting the emitted light into biological tissue. In such embodiments, the photodetector may be further arranged between the third and fourth LEDs, such that a third distance separating the photodetector and the third LED is substantially greater than or substantially less than a fourth distance separating the photodetector and the fourth LED. In some embodiments, the first LED and the third LED may be arranged on one side of the photodetector, and the second LED and the fourth LED may be arranged on an opposite side of the photodetector. In some embodiments, the first distance may be substantially equal to the third distance, and the second distance may be substantially equal to the fourth distance. In other embodiments, the first distance, the second distance, the third distance, and the fourth distance may all be substantially different from one another.

### BRIEF DESCRIPTION OF THE DRAWINGS

Other objects and advantages of the disclosure will become apparent upon reading the following detailed description and upon reference to the accompanying drawings.

FIG. 1 is a block diagram illustrating one embodiment of a biometric monitoring device comprising an improved photoplethysmographic (PPG) sensor module;

FIG. 2 is a graph illustrating certain behaviors of optical signals traveling through biological tissue;

FIG. 3 is a simplified block diagram illustrating a PPG sensor module having an optimized optical arrangement, according to a first embodiment;

FIG. 4 is a simplified block diagram illustrating a PPG sensor module having an optimized optical arrangement, according to a second embodiment;

FIG. 5A is a simplified block diagram illustrating a PPG sensor module having an optimized optical arrangement, according to a third embodiment;

FIG. 5B is a simplified block diagram illustrating a PPG sensor module having an optimized optical arrangement, according to a fourth embodiment;

FIG. 6 is a simplified block diagram illustrating a PPG sensor module having an optimized optical arrangement, according to a fifth embodiment; and

FIG. 7 is a simplified block diagram illustrating a PPG sensor module having an optimized optical arrangement, according to a sixth embodiment.

While the PPG sensor module and biometric monitoring device disclosed herein is susceptible to various modifications and alternative forms, specific embodiments thereof are shown by way of example in the drawings and will herein be described in detail. It should be understood, however, that the drawings and detailed description thereto are not intended to limit the disclosure to the particular form disclosed, but on the contrary, the intention is to cover all modifications, equivalents and alternatives falling within the spirit and scope of the present disclosure as defined by the appended claims.

### DETAILED DESCRIPTION OF PREFERRED EMBODIMENTS

The present disclosure is directed to biometric monitoring devices, which are typically (although not exclusively) designed to be worn by a user continuously, intermittently or during certain activities (e.g., while exercising). Wearable biometric monitoring devices are usually small in size, so as to be relatively unobtrusive for the wearer, and are typically designed to be worn for long periods of time without discomfort. These devices may be worn on a variety of different body parts including, for example, a user's wrist, arm, ankle, leg or chest, without interfering with the user's range of motion or daily activities. As such, a wearable biometric monitoring device may be provided in a variety of form factors including various configurations of bands or straps, which are sized to accommodate a user's wrist, arm, ankle, leg or chest, and configured to compress the device against the user's skin. Alternatively, a wearable biometric monitoring device may be attached to a user's clothing, or may be inserted into a wearable sleeve, sock or compression band that compresses the device against the user's skin.

When worn, a biometric monitoring device may collect and/or derive data corresponding to the wearer's physiological state, physical state and/or surrounding environment. As such, a biometric monitoring device may include a variety of biometric and environmental sensors for collecting and/or deriving data pertaining to heart rate, blood pressure, oxygen saturation, respiration rate, skin and/or body temperature, calories burned, floors ascended/descended, ambulatory speed and/or distance traveled, light exposure, geographic location, heading and/or elevation, etc. In addition to one or more biometric and environmental sensors, a biometric monitoring device may also include circuitry for sampling, digitizing and/or filtering the sensor signals, a processor or other control circuitry for controlling sensor functionality, processing the data collected by the sensors and/or deriving information pertaining the wearer's physiological state, physical state and/or environment, and a memory for storing program instructions executable by the processor for implementing such functionality and/or for temporarily or persistently storing the collected/processed/derived data. In some cases, a biometric monitoring device may also include a user interface for displaying the collected/processed/derived data and/or for displaying user configuration options. Additionally or alternatively, a biometric monitoring device may include a communication interface (e.g., wired or wireless) for communicating with a client or external device.

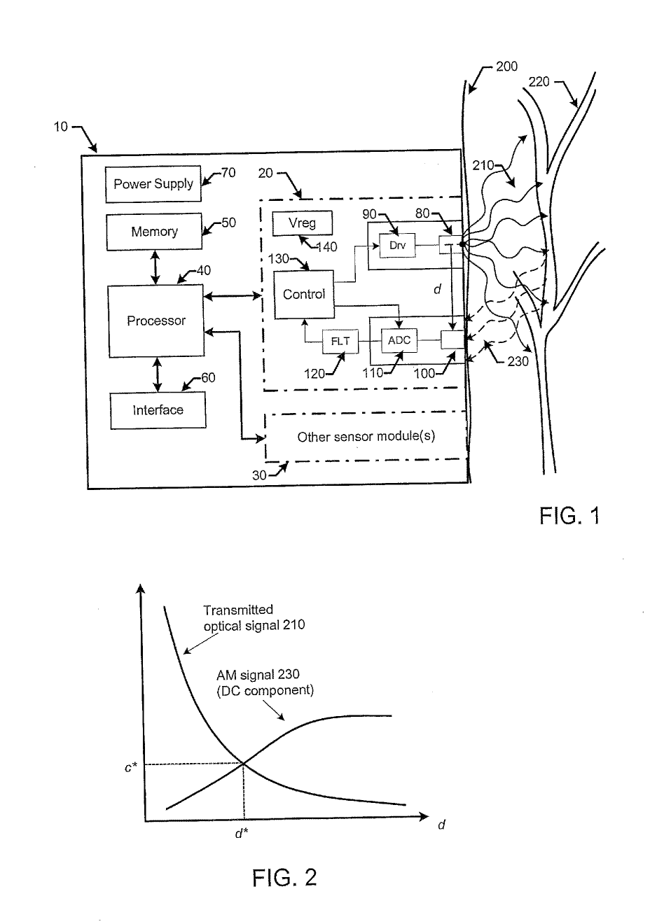

FIG. 1 is a block diagram illustrating various components of a biometric monitoring device, according to one exemplary embodiment. Although certain components of a biometric monitoring device are illustrated and described herein for purposes of demonstrating various features and functionality of the claimed device, one skilled in the art would understand and appreciate that additional and/or alternative components may be included within the biometric monitoring device shown in FIG. 1 without departing from the scope of the invention.

As shown in FIG. 1, biometric monitoring device 10 generally comprises one or more sensor modules 20, 30, a processor 40, a memory 50, an interface 60 and a power supply 70. The one or more sensor modules 20, 30 are generally configured for collecting data pertaining to a wearer's physiological state, physical state and/or surrounding environment. Processor 40 is generally configured for controlling the functionality of the biometric monitoring device 10, processing the data collected by the sensor modules and/or deriving information pertaining to the wearer's physiological state, physical state and/or environment based on the collected data. Memory 50 is generally configured for storing program instructions, which are executable by the processor 40 for implementing such functionality, and/or for temporarily or persistently storing the collected, processed and/or derived data. In some embodiments, interface 60 may be a user interface, which is configured for displaying the collected, processed and/or derived data, and/or for displaying user configuration options. In other embodiments, interface 60 may additionally or alternatively comprise a communication interface (e.g., wired or wireless communication interface) for communicating the collected, processed and/or derived data to an external device or application. Power supply 70 may comprise a battery for supplying power to the components of the biometric monitoring device 10, and in some cases, may comprise additional circuitry for regulating or rectifying the current or voltage provided by the battery to one or more components of the biometric monitoring device 10. In some embodiments, power supply 70 may comprise additional circuitry for charging a rechargeable battery.

In the particular embodiment shown in FIG. 1, biometric monitoring device 10 includes a photoplethysmographic (PPG) sensor module 20, and optionally, one or more additional sensor modules 30. Examples of additional sensor modules 30 that may be included within biometric monitoring device include, but are not limited to, a temperature sensor, an electrocardiographic sensor (ECG or EKG), a skin impedance sensor, a skin galvanic response sensor (DC resistance of skin), a motion detection sensor (e.g., an accelerometer, gyroscopic sensor, or global positioning system, GPS), an ambient light sensor, etc.

As known in the art, PPG sensor modules are generally configured for monitoring volumetric changes of an organ (e.g., an artery or arteriole). With each cardiac cycle, the heart pumps blood to the periphery of the body. Although the pressure pulse is somewhat damped by the time it reaches the skin, it is enough to distend the arteries and arterioles in the subcutaneous tissue. PPG sensor modules measure changes in volume caused by the blood pressure pulse by illuminating the skin with light and measuring the amount of light reflected back (or, optionally, transmitted through) to the sensor module. When configured for detecting hear rate, a PPG sensor module may transmit an optical signal at a wavelength and strength sufficient to reach subcutaneous tissue layers, and may receive an amplitude modulated (AM) optical signal reflected back from an artery or arteriole in the subcutaneous tissue. Each cardiac cycle appears as a peak in the AM optical signal detected by the PPG sensor module, and heart rate is typically determined by monitoring or counting the number of peaks in the AM optical signal over time.

PPG sensor modules are often referred to as optical heart rate monitors. However, because blood flow to the skin can be modulated by multiple other physiological systems, PPG sensor modules can also be used to monitor respiration and other circulatory conditions, such as blood pressure, blood volume, arterial stiffness, oxygen saturation, blood glucose levels, cardiac rhythm, hypovolemia and hypervolemia, among others.

FIG. 1 illustrates exemplary components that may be included within a PPG sensor module 20. In general, PPG sensor module 20 includes an emitter module comprising one or more LEDs 80 and associated driver(s) 90 for transmitting light 210 into the subcutaneous tissue of the wearer's skin 200. Since the purpose of the PPG sensor module 20 is to measure or monitor heart rate, the light 210 is preferably transmitted into a portion of the subcutaneous tissue relatively near an artery, arteriole or capillary 220. Although optical signal 210 is attenuated as it travels through the tissue, a portion of the transmitted optical signal 210 will be reflected from pulsating artery 220 back to PPG sensor module 20 as a reflected, amplitude modulated (AM) optical signal 230, where it is received by a detector module comprising one or more photodetectors 100, analog-to-digital converter(s) (ADCs) 110 and optionally filter(s) 120. In addition to the emitter and detector modules, PPG sensor module 20 may also include a controller 130 and voltage regulator 140. The controller 130 is coupled for receiving control data from processor 40 for controlling the emitter and detector modules, and also for receiving the digitized (and possibly filtered) signals from the detector module. The voltage regulator 140 may be included within the PPG sensor module 20 for regulating a supply voltage provided to one or more components included within the PPG sensor module 20.

In some embodiments, the wavelength of the light 210 transmitted by the one or more LEDs 80 and/or the wavelength of the light 230 detected by the one or more photodetectors 100 may be specific to a particular type of physiological and/or environmental data to be collected. When collecting and/or deriving data pertaining to heart rate, for example, the emitter module may include one or more green LEDs, which are configured for emitting light in a wavelength range extending between about 495 nm and 570 nm. In some embodiments, the emitter module may additionally or alternatively include one or more red LEDs, which are configured for emitting light in a wavelength range extending between about 620 nm and 750 nm, and one or more infrared LEDs, which are configured for emitting light in a wavelength range extending between about 700 nm and 1 mm, when collecting and/or deriving data pertaining to oxygen saturation.

Alternative light sources configured for emitting light in other wavelength ranges may also be included within the emitter module of the PPG sensor module 20. In some embodiments, for example, one or more blue LEDs configured for emitting light in a wavelength range extending between about 380 nm and 450 nm may be included within the emitter module. Since optical signals in the blue wavelength range do not penetrate into the subcutaneous layer, the blue light reflected back to the detector module may have little or no heart rate information. In some embodiments, the reflected blue light may be used as a reference signal for reducing or eliminating motion artifacts or other noise components in the reflected green light signal, which is detected by the photodetectors 100. Additional details for doing so are disclosed in U.S. patent application Ser. No. 14/872,605, which is commonly assigned and incorporated herein by reference in its entirety.

In some embodiments, the one or more photodetectors 100 may be broad spectrum photodetector(s), which are configured for detecting reflected light 230 within a broad range of wavelengths (including, e.g., ultra-violet, visible and infrared wavelengths). In other embodiments, the detector module may comprise one or more photodetectors 100, which are configured for detecting more specific wavelengths of light (e.g., wavelengths within only the blue spectrum, only the green spectrum, only the red spectrum or only the infrared spectrum). Alternatively, one or more optical filters (not shown) may be included within the detector module for narrowing the wavelength detection range of one or more broad spectrum photodetectors 100. In preferred embodiments, the one or more photodetectors 100 may comprise silicon photodiode(s), however, LEDs configured as photodetectors may be used in other embodiments.

The strength of the optical signal 230 received by the photodetector(s) 100 is dependent on many factors, including but not limited to, the intensity and wavelength of the transmitted optical signal 210, the composition and/or color of the wearer's skin 200, and the physical location of the biometric monitoring device 10 on the wearer's body. For example, the intensity and wavelength of the transmitted optical signal 210, and to some extent, the composition and/or color of the wearer's skin 200, determine the degree to which the optical signal is attenuated as it travels through the cutaneous and subcutaneous tissues. In addition, since blood flow signals are substantially weaker in peripheral areas of the body (e.g., the wrist, ankle and other areas having poor vasculation), it is generally more difficult to obtain accurate heart rate signals when biometric monitoring device 10 is placed on such peripheral locations.

In traditional biometric monitoring devices, it is relatively common to mount or embed a photodetector and one or more LEDs on an external surface of the monitoring device, which is designed to be adjacent to the wearer's skin. In some devices, the photodetector may be arranged between a pair of LEDs for detecting reflected light, and in some cases, one or more light shields and/or light guides may be used to block or reduce detection of the light, which is directly transmitted by the LEDs to the photodetector. However, traditional biometric monitoring devices often fail to take into account the size, shape, orientation and placement of the photodetector relative to the LEDs when designing the PPG sensor module. Through extensive experimentation and analysis, the present inventors have recognized that measurement accuracy can be significantly improved by optimizing the optical arrangement of the LED(s) and photodetector(s) included within the PPG sensor module 20. In addition to improving measurement accuracy, optimizing the optical arrangement of the LED(s) and photodetector(s) results in significant power savings by enabling the drive currents and/or duty cycle supplied to the LED(s) to be reduced without impacting the signal-to-noise ratio of the measurement signal.

The optical arrangement can be optimized, at least in part, by optimizing the distance (d) separating an LED 80 and a respective photodetector 100. As shown in FIGS. 1 and 2, the intensity of the transmitted optical signal 210 drops off quickly as the distance (d) between the LED 80 and the photodetector 100 increases. In a typical human wrist, for example, diffusion in the tissue may cause the light carrier energy to drop off every millimeter (mm) of distance (d) separating the LED 80 and photodetector 100. Because low carrier energy creates more shot noise in the detected signal, traditional biometric monitoring devices tend to drive the LED(s) harder to improve the signal-to-noise ratio. This is undesirable, since it increases the power consumption and decreases the efficiency of the LEDs.

FIG. 2 further shows that, while the carrier energy decreases, the DC component or magnitude of the reflected signal 230 increases somewhat linearly (up to a point) as the distance (d) between the LED 80 and the photodetector 100 increases. Therefore, an optimal distance (d*) between an LED 80 and a respective photodetector 100 may be chosen, so as to maximize the amount of reflected light 230 detected by the photodetector 100 for a given carrier strength (c*), which has been predetermined to minimize or provide an acceptable amount of shot noise.

According to one embodiment, the optimal distance (d*) between a green LED 80 and a respective photodetector 100 may range between about 0.5 mm and about 8 mm. While the lower end of this range (e.g., about 0.5 mm to about 2 mm) may result in relatively low modulation intensity 230, it may be preferred for darker skin tones, which add additional tissue entrance and exit attenuation. Likewise, while the upper end of this range (e.g., about 2 mm to about 8 mm) may be subject to increased shot noise (due to lower carrier signal strength 210), it may be preferred for lighter skin tones, which don't present as much attenuation to the modulated signal 230. Through extensive experimentation and analysis, the present inventors have found that an optimal distance (d*) ranging between about 1 mm and about 5 mm, and more preferably, between about 1.5 mm and about 3 mm, has been found to provide an acceptable compromise between shot noise and modulation intensity for a variety of different skin types. However, since the optimal distance (d*) is also dependent the wavelength of the transmitted light 210, different ranges may be appropriate when different colors of LEDs (e.g., blue, red or infrared) are used to transmit light into the wearer's skin.

According to one embodiment, measurement accuracy is improved in PPG sensor module 20 by arranging an LED 80 and a respective photodetector 100 at an optimal distance (d*), which maximizes the amount of reflected light 230 detected by the photodetector 100 for a given carrier strength (c*). Because shot noise is minimized (or at least reduced to an acceptable level) at the optimal distance, the LED(s) don't have to be driven as hard as in traditional biometric monitoring devices, thus providing power savings and improving LED efficiency. However, since the optimal distance (d*) between an LED 80 and a respective photodetector 100 is dependent on other factors, such as the wavelength of the transmitted light 210, the composition and color of the wearer's skin, and the depth of the artery being monitored, some embodiments may include a plurality of LED/photodetectors pairs within the PPG sensor module 20, and an optimal distance (d*) may be determined separately for each LED/photodetector pair. By including a plurality of LED/photodetector pairs within the PPG sensor module 20, each pair potentially separated by a different optimal distance (d*), PPG sensor module 20 provides signal diversity and may significantly improve measurement accuracy for a variety of different skin types and measurement locations (e.g., wrist, upper arm, ankle, etc.) by enabling the reflected signal 230 with the best signal-to-noise ratio to be used for monitoring heart rate.

Separation distance is not the only factor that should be considered when optimizing the optical arrangement of the LED(s) and photodetector(s). When pressed against the wearer's skin, LED(s) 80 radiate light substantially omni-directionally into the surrounding tissue, and at least a portion of this light is reflected back to the PPG sensor module 20 in a similar pattern. In an ideal situation, an optimally efficient sensor module would position an LED in the center of a relatively thin photodetector ring surrounding the LED, so as to maximize the collection angle of the reflected light (i.e., provide a 360° collection angle) that can be detected by the photodetector. Since a 360° ring is impractical, a more realistic arrangement is to maximize the collection angle (x) of reflected light that can be detected by the one or more photodetectors 100 within the space and cost constraints of the PPG sensor module 20. This is achieved in the present disclosure by utilizing at least one elongated photodetector (e.g., a photodetector whose length is substantially greater than its width, or vice versa) and orienting the at least one elongated photodetector, such that a longer dimension (e.g., a length) or longer edge of the photodetector is arranged facing the LED and/or arranged substantially perpendicular to a radiation pattern of the reflected light.

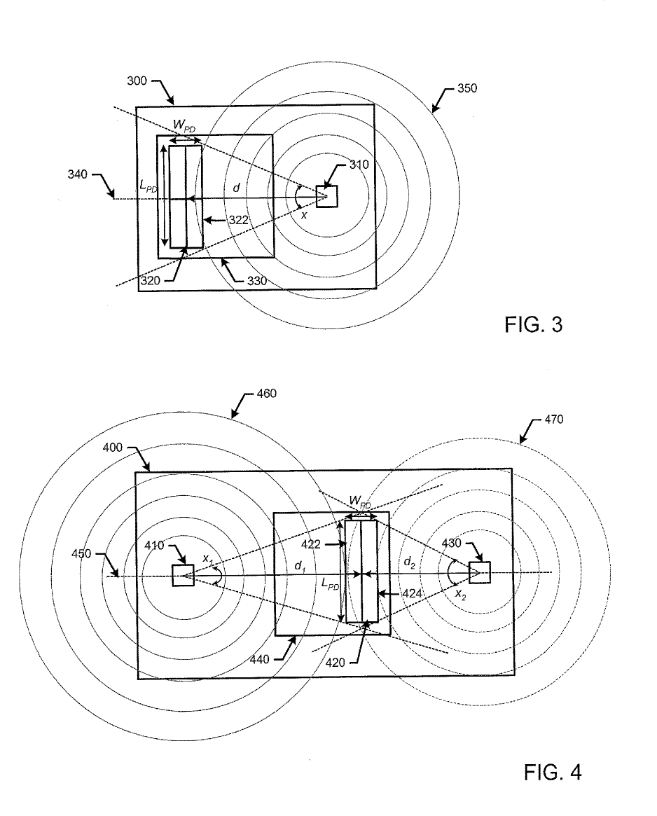

FIGS. 3-7 illustrate various preferred embodiments of a PPG sensor module having an optimized optical arrangement. In addition to arranging at least one LED and at least one respective photodetector at an optimal distance (d), FIGS. 3-7 show that the optical arrangement can be further optimized by maximizing the collection angle (x) of reflected light, which can be detected by the photodetector.

A first preferred embodiment of a PPG sensor module 300 is shown in FIG. 3 as including a single LED 310 and a single photodetector 320. LED 310 and photodetector 320 may be generally configured for emitting and detecting light, as described above for LED 80 and photodetector 100. In the embodiment of FIG. 3, reference numeral 330 represents an approximate placement of the remaining PPG sensor module components (e.g., LED driver(s) 90, ADC(s) 110, filter(s) 120, control circuitry 130 and voltage regulator 140) shown in FIG. 1. The approximate placement of the remaining PPG sensor module components is likewise represented by reference numerals 440, 560, 565, 570, 575 and 680 in FIGS. 4-7. However, a skilled artisan would understand that the remaining PPG sensor module components are not necessarily limited to the placements shown in FIGS. 3-7.

Unlike the square photodetectors typically used in conventional PPG sensor modules, photodetector 320 is an elongated photodetector having a substantially longer length (L_{PD}) than width (W_{PD}). According to one embodiment, the length (L_{PD}) of the elongated photodetector 320 may be approximately 1.5 to approximately 5 times larger than the width (W_{PD}) of the elongated photodetector 100. More preferably, the length (L_{PD}) of the elongated photodetector 320 may be approximately 2 to approximately 4 times larger than the width (W_{PD}) of the elongated photodetector 320. In one example, the length (L_{PD}) of the elongated photodetector 320 is approximately 2 mm and the width (W_{PD}) of the elongated photodetector 320 is approximately 0.5 mm. However, photodetector 320 is not limited to the exemplary dimensions described herein, and may comprise substantially any dimensions that fit within the space constraints of PPG sensor module 300 and provide an elongated photodetector having a longer dimension (e.g., L_{PD}) or a longer edge at least 1.5 times larger than a shorter dimension (e.g., W_{PD}) or shorter edge of the photodetector. Compared to square photodetectors, an elongated photodetector 320 is configured to collect more reflected light when properly oriented.

As noted above with respect to FIGS. 1 and 2, the magnitude of the transmitted light 210 decreases, and the magnitude of the reflected light 230 increases, as the distance (d) between the LED 310 and the photodetector 320 increases. Therefore, the optimal distance (d) between LED 310 and photodetector 320 may be chosen, so as to maximize the magnitude of the reflected light impinging upon the photodetector 320, while reducing or maintaining an acceptable amount of shot noise. The optimal distance (d) is dependent, at least in part, on the size of the photodetector 320 and the space constraints of the PPG sensor module 300 and may be chosen, so as to maximize the amount of reflected light detected by the photodetector 320 for a given carrier strength, which has been predetermined to minimize or provide an acceptable amount of shot noise. As noted above, the optimal distance (d) between LED 310 and photodetector 320 may range between about 0.5 mm and about 8 mm, about 1 mm and about 5 mm, or about 1.5 mm and about 3 mm.

In order to maximize the collection angle (x) of reflected light, the elongated photodetector 320 is preferably oriented with its longer dimension (L_{PD}), or longer edge 322, oriented towards or facing the LED 310. More specifically, the elongated photodetector 320 is preferably positioned so that a line 340 passing through a center point of the LED 310 in the plane of the PPG sensor module 300 substantially bisects its longer dimension (L_{PD}) or longer edge 322. Stated another way, the elongated photodetector 320 is preferably positioned so that the line 340 passes through the center point of LED 310 and the center point of photodetector 320. In doing so, the elongated photodetector 320 may be arranged so that the longer dimension (L_{PD}) is substantially perpendicular to the radiation pattern 350 of transmitted light, so that more of the reflected light can be collected along its longer edge 322.

In FIG. 3, each circle in radiation pattern 350 represents a different magnitude of transmitted light arriving at the elongated photodetector 320. Moving outward from LED 310, each subsequent circle in radiation pattern 350 represents a substantially lower magnitude of transmitted light 210, yet higher magnitude of reflected light 230 arriving at the elongated photodetector 320. In the present disclosure, the collection angle (x) can be maximized by centering the elongated photodetector 320 about line 340 and orienting the longer dimension (L_{PD}) or longer edge 322 of the elongated photodetector 320 along one of the arcs in the transmitted radiation pattern 350. According to one example, a 0.6 mm by 1.8 mm photodetector 320 spaced approximately 2 mm from LED 310 may have a collection angle (x) of approximately 47°. The strength of the measurement signal is improved by orienting the elongated photodetector 320 in such a manner, since more of the reflected light impinges upon the longer edge 322 of the photodetector.

FIG. 4 illustrates another embodiment of a PPG sensor module 400 having an optimized optical arrangement. Similar to PPG sensor module 300, PPG sensor module 400 improves the signal-to-noise ratio of the measurement signal by positioning an elongated photodetector 420 at an optimal distance (d) and orienting the photodetector, so as to maximize the collection angle (x) of reflected light. The PPG sensor module 400 shown in FIG. 4 differs from the one shown in FIG. 3, however, by arranging the elongated photodetector 420 between two LEDs 410 and 430. In some embodiments, LEDs 410 and 430 may be configured for emitting the same wavelength of light (e.g., light within a green wavelength range). In other embodiments, LEDs 410 and 430 may be configured for emitting substantially different wavelengths of light. In one example, LED 410 may be configured emitting light with a green wavelength range, while LED 430 may be configured for emitting light within a blue, red or infrared wavelength range.

The elongated photodetector 420 may be generally dimensioned, as discussed above with respect to FIG. 3, and may be arranged between LEDs 410 and 430 to alternately collect light, which is emitted by either LED 410 or LED 430 into the biological tissue and reflected back to the PPG sensor module 400. For example, control signals provided to, or generated by, the control circuitry within the PPG sensor module circuitry 440 may enable the photodetector 420 collect light, which is emitted by LED 410 and reflected back to the PPG sensor module 400 during a first time frame. During a second time frame, subsequent control signals may enable the photodetector 420 to collect light, which is emitted by LED 430 and reflected back to the PPG sensor module 400.

As in the previous embodiment, the elongated photodetector 420 is preferably: (a) oriented with its longer edges 422 and 424 respectively facing or oriented towards the LEDs 410 and 430, and (b) positioned so that a line 450 passing through a center point of the LEDs 410 and 430 in the plane of the PPG sensor module 400 substantially bisects its longer dimension (L_{PD}) or longer edges 422 and 424. In other words, the elongated photodetector 420 is preferably positioned so that the line 450 passes through the center points of LEDs 410 and 430 and also through a center point of photodetector 420. In doing so, the photodetector 420 is preferably arranged so that the longer dimension (L_{PD}) is substantially perpendicular to the radiation patterns 460 and 470 of reflected light, so that more of the reflected light can be collected along it's longer edges 422 and 424. As noted above, positioning the photodetector 420 in such a manner improves the strength of the measurement signal by maximizing the collection angles (x₁ and x₂) of the reflected light that can be detected by the photodetector 420.

In the embodiment of FIG. 4, LEDs 410 and 430 are separated from photodetector 420 by different distances (d₁ and d₂). The distance (d₁) between LED/photodetector pair 410/420 may be chosen to maximize the amount of reflected light detected by photodetector 420 for a given carrier strength, which has been predetermined to minimize or provide an acceptable amount of shot noise. On the other hand, the distance (d₂) between LED/photodetector pair 430/420 may be chosen, so as to provide diversity for the heart rate measurement signal and to account for variations in skin color, skin composition and measurement location (e.g., wrist, upper arm, ankle, etc.).

In the exemplary embodiment shown in FIG. 4, the distance (d₂) between LED 430 and photodetector 420 is significantly smaller than the distance (d₁) between LED 410 and photodetector 420. For example, the distance (d₂) between LED/photodetector pair 430/420 may be approximately 2.0 mm, and the distance (d₁) between LED/photodetector pair 410/420 may be approximately 3.0 mm, in one embodiment. At these exemplary separation distances, photodetector 420 may have a collection angle (x₁) of approximately 33° for detecting reflected light originating from LED 410 and a collection angle (x₂) of approximately 49° for detecting reflected light originating from LED 430.

By arranging LED 430 closer to photodetector 420 (d₂<d₁), the magnitude of the light reflected back to the photodetector is smaller; however, since the collection angle is larger (x₂>x₁), the amount of reflected light impinging upon the photodetector 420 may provide a better signal-to-noise ratio for certain skin types and measurement locations. In other embodiments (not shown in FIG. 4), signal diversity may be provided by arranging LED 430 farther from photodetector 420 (d₂>d₁). In either case, by including a plurality of LED/photodetector pairs (e.g., 410/420 and 430/420) and separating each pair by a different distance (d₁ and d₂), PPG sensor module 400 not only provides signal diversity, but improves measurement accuracy for a variety of different skin types and measurement locations by enabling the sensed signal with the best signal-to-noise ratio to be used for monitoring heart rate.

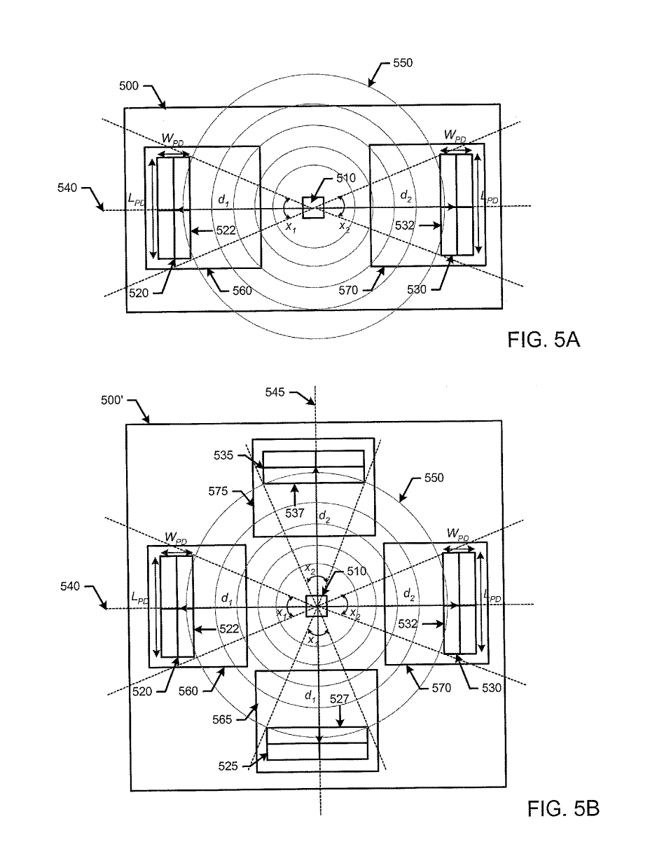

FIGS. 5A-5B illustrate yet another embodiment of a PPG sensor module 500/500′ having an optimized optical arrangement. In FIG. 5A, a single LED 510 is centrally arranged between a pair of elongated photodetectors 520 and 530. In FIG. 5B, LED 510 is centrally arranged between two pairs of elongated photodetectors 520/530 and 525/535. It is noted that although FIGS. 5A and 5B illustrate the LED 510 as being centrally arranged between 2 and 4 photodetectors, respectively, a skilled artisan would recognize that the PPG sensor module 500 may be further optimized by arranging more than 4 photodetectors around the LED 510. However, the skilled artisan may recognize that while including additional photodetectors may further optimize the optical arrangement and improve the accuracy of the detected measurement signal, the additional photodetectors may increase the cost and size of the PPG sensor module. As shown in FIG. 5B, for example, PPG sensor module 500′ may be slightly larger in at least one dimension (e.g., length) to accommodate the additional photodetectors 525/535.

Although the green wavelength range is preferable for monitoring heart rate, LED 510 may be generally configured for emitting light within any wavelength range discussed above. The elongated photodetectors 520, 525, 530 and 535 may be generally dimensioned as discussed above. Each of the photodetectors 520, 525, 530 and 535 is also preferably: (a) oriented with one of its longer edges (522, 527, 532, 537) facing or oriented towards LED 510, and (b) positioned so that a line 540 passing through a center point of LED 510 in the plane of the PPG sensor module 500 substantially bisects the longer dimension (L_{PD}) or longer edge of the photodetector. In other words, LED 510 is preferably arranged between elongated photodetectors 520 and 530, such that the line 540 passing through the center point of LED 510 also passes through the center points of photodetectors 520 and 530 (FIGS. 5A and 5B). Likewise, LED 510 is preferably arranged between elongated photodetectors 525 and 535, such that the line 545 passing through the center point of LED 510 also passes through the center points of photodetectors 525 and 535 (FIG. 5B). In doing so, each photodetector may be arranged with its longer dimension (L_{PD}) substantially perpendicular to the radiation pattern 550 of reflected light, so that more of the reflected light can be collected along its longer edge (522, 527, 532, 537).

Similar to the previous embodiment, PPG sensor module 500 provides signal diversity and improves measurement accuracy by providing a plurality of LED/photodetector pairs (e.g., 510/520, 510/525, 510/530 and 510/535). The distance separating the LED/photodetector pairs may be substantially the same (e.g., d₁ may be substantially =d₂), or may be substantially different (e.g., d₁ may be substantially > or <d₂). Likewise, the collection angle of reflected light that can be detected by the photodetectors 520, 525, 530 and 535 may be substantially the same (e.g., x₁ may be substantially =x₂), or may be substantially different (e.g., x₁ may be > or <x₂). According to one embodiment, d₁ and d₂ are both approximately equal to 3 mm and x1 and x2 are both approximately equal to 33°. Even if d₁=d₂ and x₁=x₂, PPG sensor module 500 may still provide signal diversity and improve measurement accuracy simply by providing a different signal path between LED/photodetector pairs 510/520, 510/525, 510/530 and 510/535, which may account for tissue unevenness and other variations.

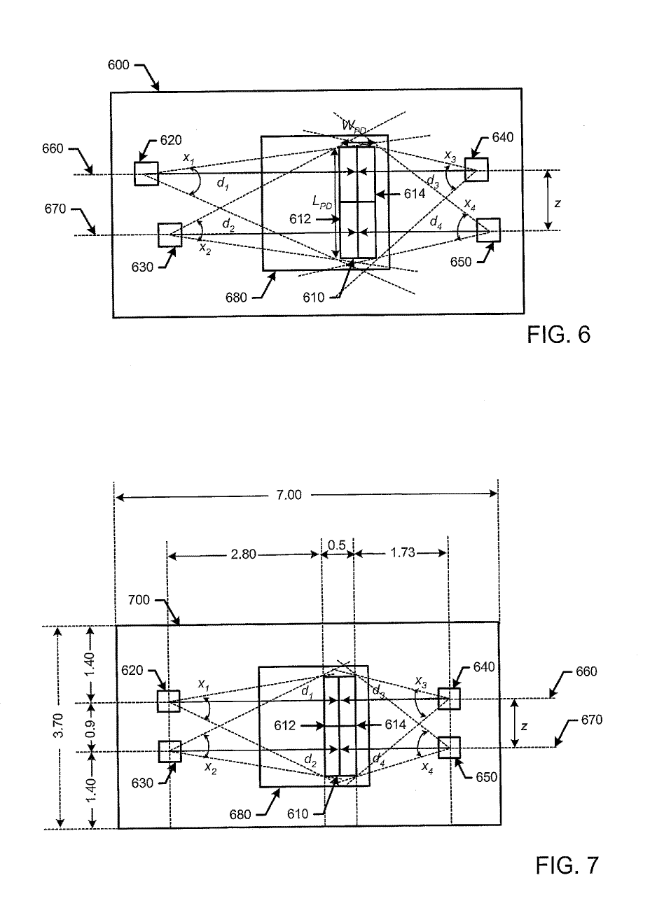

Additional embodiments of a PPG sensor module 600/700 having an optimized optical arrangement are illustrated in FIGS. 6 and 7. Like the embodiment shown in FIG. 4, PPG sensor module 600/700 positions an elongated photodetector 610 between a plurality of LEDs. In PPG sensor module 600/700, however, the elongated photodetector 610 is positioned between four LEDs 620, 630, 640, and 650. Although four LEDs are shown in FIGS. 6 and 7, a skilled artisan would understand that a greater or lesser number of LEDs could be included within PPG sensor module 600/700 depending on the cost and size constraints of the PPG sensor module.

In general, LEDs 620, 630, 640, and 650 may each be configured for emitting light within any wavelength range discussed above. In some embodiments, LEDs 620, 630, 640, and 650 may each be configured for emitting light within the same wavelength range (e.g., each may emit light within a green wavelength range). In other embodiments, at least one of the LEDs may be configured for emitting light within a green wavelength range, while one or more of the remaining LEDs may be configured for emitting one or more different wavelengths of light (e.g., light within a blue, red, and/or infrared wavelength range).

In some embodiments, photodetector 610 may be a broad spectrum photodetector, which is configured for detecting reflected light within a broad range of wavelengths (including, e.g., ultra-violet, visible and infrared wavelengths). In other embodiments, photodetector 610 may be configured for detecting more specific wavelengths of light (e.g., wavelengths within only the blue spectrum, only the green spectrum, only the red spectrum or only the infrared spectrum). Regardless, photodetector 610 may generally be used for detecting reflected green light for the purpose of obtaining heart rate information. In some embodiments, photodetector 610 may additionally be used for detecting reflected blue light for the purpose of obtaining a reference signal for reducing noise components in the reflected green light signal, and/or for detecting reflected red and infrared light for the purpose of obtaining information pertaining to oxygen saturation. In preferred embodiments, photodetector 610 is a silicon photodiode. However, an LED configured as a photodetector may be used in other embodiments.

As in the previous embodiments, photodetector 610 is an elongated photodetector having a substantially longer length (L_{PD}) than width (W_{PD}). In this embodiment, however, the length (L_{PD}) of the elongated photodetector 610 may be approximately 1.5 to approximately 8 times larger than the width (W_{PD}) of the elongated photodetector 610 to accommodate the additional LEDs. As before, the elongated photodetector 610 is preferably: (a) oriented with its longer edges 612 and 614 oriented towards or facing the LEDs 620, 630, 640, and 650, and (b) arranged so that the longer dimension (L_{PD}) is substantially perpendicular to the radiation patterns of reflected light (removed from FIGS. 6 and 7 for purposes of drawing clarity), so that more of the reflected light can be collected along its longer edges 612 and 614. As noted above, positioning the photodetector in such a manner improves the strength of the measurement signal by maximizing the collection angles (x₁, x₂, x₃ and x₄) of the reflected light that can be detected by photodetector 610.

Unlike the previous embodiments, photodetector 610 is not positioned so that a line passing through a center point of the LEDs 620, 630, 640, and 650 in the plane of the PPG sensor module 600 substantially bisects its longer dimension (L_{PD}) or longer edges. In order to accommodate multiple LEDs on either side of the photodetector, photodetector 610 is preferably positioned so that a line 660 passing through a center point of LEDs 620 and 640 in the plane of the PPG sensor module 600 passes through a top half portion of its longer dimension (L_{PD}), while another line 670 passing through a center point of LEDs 630 and 650 in the plane of the PPG sensor module 600 passes through a bottom half portion of its longer dimension (L_{PD}). The separation distance (z) between LEDs on similar sides of the photodetector 610 (e.g., the separation distance between LEDs 620/630 and LEDs 640/650) may be as close as possible to maximize the collection angle (x). However, assembly requirements may increase the separation distance (z) up to approximately the length of the photodetector.

Similar to the embodiments shown in FIGS. 4 and 5, PPG sensor module 600/700 provides signal diversity and improves measurement accuracy by providing a plurality of LED/photodetector pairs (e.g., 620/610, 630/610, 640/610 and 650/610). In some cases, the distance separating each LED/photodetector pair may be substantially the same (e.g., d₁=d₂=d₃=d₄). In FIG. 6, however, each LED/photodetector pair is separated by a different distance (e.g., d₁≠d₂≠d₃≠d₄), and each LED/photodetector pair comprises a substantially different collection angle of reflected light (e.g., x₁≠x₂≠x₃≠x₄). In one example, d₁ may be approximately 4 mm, d₂ may be approximately 3 mm, d₃ may be approximately 2 mm, and d₄ may be approximately 1.5 mm. At these separation distances, a 1.8 mm by 0.6 mm (length by width) photodetector 610 may have a collection angle x₁ of about 25°, a collection angle x₂ of about 33°, a collection angle x₃ of about 49°, and a collection angle x₄ of about 62°. Regardless of the exact dimensions used, the PPG sensor module 600 shown in FIG. 6 may provide maximum signal diversity by providing a substantially different signal path between each LED/photodetector pair.

In FIG. 7, the LED/photodetector pairs (e.g., 620/610 and 630/610) on one side of the photodetector 610 are separated by a first common distance (e.g., d₁=d₂), while the LED/photodetector pairs (e.g., 640/610 and 650/610) on the opposite side of the photodetector 610 are separated by a second common distance (e.g., d₃=d₄), which differs from the first common distance (e.g., d₃, d₄≠d₁, d₂). Since d₁ is substantially =d₂, the collection angle of the reflected light that is detected by photodetector 610 from light emitted by LEDs 620 and 630 may also be substantially equivalent (e.g., x₁ may be substantially =x₂). Likewise, since LED/photodetector pairs 640/610 and 650/610 are also separated by a common distance (e.g., d₃=d₄), these pairs also comprise a similar collection angle (e.g., x₃ may be substantially =x₄). However, since the second common distance separating LED/photodetector pairs 640/610 and 650/610 is substantially less than the first common distance separating LED/photodetector pairs 620/610 and 630/610 (e.g., d₃<d₁), collection angles x₃ and x₄ may be substantially greater than collection angles x₁ and x₂.

FIG. 7 illustrates exemplary dimensions for PPG sensor module 700. According to the illustrated embodiment, the distance (d₁) between the center point of LED 620 and a first long edge 612 of photodetector 610, and the distance (d₂) between the center point of LED 630 and the first long edge 612 may be approximately 2.80 mm. On the other side of the photodetector, the distance (d₃) between the center point of LED 640 and a second long edge 614 of photodetector 610, and the distance (d₄) between the center point of LED 650 and the second long edge 614 may be approximately 1.73 mm. LEDs on similar sides of the photodetector 610 (e.g., LEDs 620/630 and LEDs 640/650) are separated by approximately 0.9 mm in the example embodiment shown in FIG. 7.

In FIG. 7, photodetector 610 is an elongated photodetector having a length (L_{PD}) of approximately 1.8 mm and a width (W_{PD}) of approximately 0.6 mm. Photodetector 610 is positioned so that the line 660 passing through the center point of LEDs 620 and 640 passes through a top half portion of it's longer dimension (L_{PD}), while the line 670 passing through the center point of LEDs 630 and 650 passes through a bottom half portion of it's longer dimension (L_{PD}). In the particular embodiment shown in FIG. 7, line 660 bisects the top half portion, while line 670 bisects the bottom half portion of the photodetector 610. Since d₁=d₂ and d₃=d₄, the collection angle x₁=x₂ and the collection angle x₃=x₄. In one example, collection angles x₁ and x₂ may be approximately 35° and collection angles x₃ and x₄ may be approximately 54°.

Although alternative dimensions can certainly be used, the exemplary dimensions shown in FIG. 7 have been shown to provide improvements in heart rate measurement accuracy, as well as sufficient signal diversity to accommodate a variety of different skin types and measurement locations, all within a relatively small sensor module footprint (e.g., approximately 3.70 mm by 7.00 mm). The small footprint of the PPG sensor modules 300, 400, 500, 600 and 700 described herein enables the overall size of the biometric monitoring device 10 to be reduced, which is a desirable feature in wearable devices. As noted above, optimizing the optical arrangement within the PPG sensor modules 300, 400, 500, 600 and 700 improves the signal-to-noise ratio (SNR) of the detected signal. Because the SNR is improved, the drive currents and/or duty cycle supplied to the LED(s) can be reduced without sacrificing measurement accuracy. This is particularly useful in both portable and wearable biometric monitoring devices, which benefit from the extended battery life that comes from reduced power consumption.

It will be appreciated to those skilled in the art having the benefit of this disclosure that this disclosure is believed to provide an improved biometric monitoring device that provides accurate heart rate monitoring in a space-constrained wearable device, while reducing power consumption and extending battery life. Although described herein as a portable or wearable biometric monitoring device, and more specifically, a wrist-worn biometric monitoring device, the PPG sensor modules described herein could be used in a variety of monitoring devices, including activity-tracking fitness bands and straps, pedometers and smart watches, as well as fitness equipment, bathroom scales and patient monitoring devices.

Further modifications and alternative embodiments of various aspects of the disclosure will be apparent to those skilled in the art in view of this description. For example, although the embodiments of PPG sensor modules are shown and described herein as comprising somewhere between 1-4 LEDs and somewhere between 1-2 photodetectors, a skilled artisan having the benefit of this disclosure would understand how a greater number of LEDs and/or a greater number of photodetectors may be used within the PPG sensor module without departing from the scope of the disclosure. However, a skilled artisan would also understand that a greater number of LEDs/photodetectors would increase the cost and size of the PPG sensor module, and that cost and size may dictate the number of LEDs/photodetectors ultimately included within the PPG sensor module.

It is to be understood that the various embodiments of PPG sensor modules and biometric monitoring devices shown and described herein are to be taken as the presently preferred embodiments. Elements and materials may be substituted for those illustrated and described herein, parts and processes may be reversed, and certain features of the PPG sensor modules may be utilized independently, all as would be apparent to one skilled in the art after having the benefit of this disclosure. It is intended, therefore, that the following claims be interpreted to embrace all such modifications and changes and, accordingly, the specification and drawings are to be regarded in an illustrative rather than a restrictive sense.

1. A photoplethysmographic (PPG) sensor module, comprising:
a first light emitting diode (LED) configured to emit light, wherein the first LED is positioned on the PPG sensor module to transmit the emitted light into biological tissue; and
an elongated photodetector configured to detect a portion of the light that is transmitted by the first LED into the biological tissue and reflected back to the elongated photodetector, wherein a longer dimension of the elongated photodetector is at least 1.5 times larger than a shorter dimension of the elongated photodetector, and wherein the elongated photodetector is positioned on the PPG sensor module, such that the longer dimension is facing towards the first LED.

2. The PPG sensor module as recited in claim 1, wherein the longer dimension of the elongated photodetector is approximately 1.5 to approximately 8 times larger than the shorter dimension of the elongated photodetector.

3. The PPG sensor module as recited in claim 1, wherein the elongated photodetector is further positioned on the PPG sensor module, such that a line passing through a center point of the first LED in a plane of the PPG sensor module substantially bisects the longer dimension of the elongated photodetector.

4. The PPG sensor module as recited in claim 1, further comprising a second LED, which is configured to emit light and positioned on the PPG sensor module to transmit the emitted light into the biological tissue.

5. The PPG sensor module as recited in claim 4, wherein the elongated photodetector is positioned on the PPG sensor module between the first LED and the second LED, such that a line passing through a center point of the first LED and a center point of the second LED in a plane of the PPG sensor module substantially bisects the longer dimension of the elongated photodetector.

6. The PPG sensor module as recited in claim 4, wherein the elongated photodetector is further positioned on the PPG sensor module between the first LED and the second LED, such that a line passing through a center point of the first LED and a center point of the second LED in the plane of the PPG sensor module passes through a top half portion of the longer dimension of the elongated photodetector.

7. The PPG sensor module as recited in claim 4, further comprising a third LED and a fourth LED, each of which are configured to emit light and positioned on the PPG sensor module to transmit the emitted light into the biological tissue.

8. The PPG sensor module as recited in claim 7, wherein the elongated photodetector is further positioned on the PPG sensor module between the third LED and the fourth LED, such that a line passing through a center point of the third LED and a center point of the fourth LED in the plane of the PPG sensor module passes through a bottom half portion of the longer dimension of the elongated photodetector.

9. The PPG sensor module as recited in claim 1, further comprising an additional elongated photodetector, which is configured to detect a portion of the light that is transmitted by the first LED into the biological tissue and reflected back to the additional elongated photodetector:
wherein a longer dimension of the additional elongated photodetector is at least 1.5 times larger than a shorter dimension of the additional elongated photodetector; and
wherein the additional elongated photodetector is positioned on the PPG sensor module, such that a line passing through a center point of first LED in the plane of the PPG sensor module substantially bisects the longer dimension of the elongated photodetector and the longer dimension of the additional elongated photodetector.

10. The PPG sensor module as recited in claim 9, wherein the first LED is arranged between the elongated photodetector and the additional elongated photodetector, such that a first distance separating the elongated photodetector and the first LED is substantially greater than or substantially less than a second distance separating the first LED and the additional elongated photodetector.

11. A photoplethysmographic (PPG) sensor module, comprising:
a first light emitting diode (LED) and a second LED, each configured to emit light and positioned on the PPG sensor module to transmit the emitted light into biological tissue;
a photodetector configured to detect a portion of the light, which is transmitted by at least one of the first and second LEDs into the biological tissue and reflected back to the photodetector; and
wherein the photodetector is arranged on the PPG sensor module between the first and second LEDs, such that a first distance separating the photodetector and the first LED is substantially greater than or substantially less than a second distance separating the photodetector and the second LED.

12. The PPG sensor module as recited in claim 11, wherein the first distance is approximately 2 to approximately 16 times greater than or less than the second distance.

13. The PPG sensor module as recited in claim 11, wherein the photodetector is an elongated photodetector having a longer dimension, which is at least 1.5 times larger than a shorter dimension of the elongated photodetector, and wherein the elongated photodetector is positioned on the PPG sensor module, such that a line passing through respective center points of the first and second LEDs in a plane of the PPG sensor module also passes through a center point of the photodetector.

14. The PPG sensor module as recited in claim 11, further comprising a third LED and a fourth LED, each configured to emit light and positioned on the PPG sensor module to transmit the emitted light into biological tissue, wherein the photodetector is further arranged on the PPG sensor module between the third and fourth LEDs, such that a third distance separating the photodetector and the third LED is substantially greater than or substantially less than a fourth distance separating the photodetector and the fourth LED.

15. The PPG sensor module as recited in claim 14, wherein the first LED and the third LED are arranged on one side of the photodetector and the second LED and the fourth LED are arranged on an opposite side of the photodetector.

16. The PPG sensor module as recited in claim 15, wherein the first distance is substantially equal to the third distance, and wherein the second distance is substantially equal to the fourth distance.

17. The PPG sensor module as recited in claim 15, wherein the first distance, the second distance, the third distance, and the fourth distance are all substantially different from one another.

18. The PPG sensor module as recited in claim 15, wherein the photodetector is an elongated photodetector having a longer dimension, which is at least 1.5 times larger than a shorter dimension of the elongated photodetector.

19. The PPG sensor module as recited in claim 18, wherein the elongated photodetector is further positioned on the PPG sensor module, such that a line passing through a center point of the first LED and a center point of the second LED in the plane of the PPG sensor module passes through a top half portion of the longer dimension of the elongated photodetector.

20. The PPG sensor module as recited in claim 18, wherein the elongated photodetector is further positioned on the PPG sensor module, such that a line passing through a center point of the third LED and a center point of the fourth LED in the plane of the PPG sensor module passes through a bottom half portion of the longer dimension of the elongated photodetector.
